(19) United States
(12) Patent Application Publication
(10) Pub. No.: US 2020/0205870 A1
(43) Pub. Date: Jul. 2, 2020

(54) CANNULATED FIXATION DEVICE

(71) Applicant: DEVICE SYNERGIES, PTY LTD, Matraville, Victoria (AU)

(72) Inventors: William Robert Walsh, Maroubra (AU); Ewen Laird, Matraville (AU)

(21) Appl. No.: 16/645,249

(22) PCT Filed: Sep. 5, 2018
(86) PCT No.: PCT/AU2018/050959

§ 371 Date: Mar. 6, 2020

(30) Foreign Application Priority Data
Sep. 8, 2017 (AU) 2017903656
Jun. 4, 2018 (AU) 2018901995

(51) Int. Cl. A61B 17/86

(52) U.S. Cl. CPC A61B 17/864; A61B 2017/8655; A61B 17/8615; A61B 17/863

(57) ABSTRACT

A fixation device comprising a shaft extending from a leading end to a trailing end, an aperture being eccentric from a centre of the trailing end, and, a channel extending from the aperture and along at least a portion of an external surface of the body toward the leading end, the aperture and channel being aligned to form a passage.

### TECHNICAL FIELD

This disclosure relates to fixation devices and particularly to threaded fixation devices such as screws. The disclosure further relates to fixation devices that include fastening or securing elements. In general in this specification, the devices have been described in relation to medical purposes such as bone screws used for bone to bone fixation, bone to implant fixation or bone fixation with other material such as tissues. However people skilled in the art will be aware that the fixation devices and associated elements have utility in other medical and non-medical fields whenever fixation is indicated.

### BACKGROUND

Fixation devices such as screws or plugs have long been used to affix two objects by compressing two objects against one another. In some forms these act by translating rotational motion to linear motion and compressing the objects against one another. In the case of plugs they work by using pushing or linear pressure to compress the objects against one another.

The security of the fixation between the objects can be limited by the tendency of a screw or device to back out of the fixed position over time, loosen or toggle in the fixation site due to imposed loads or the quality of the object the devices are placed in or sub-optimal placement procedures. In some circumstances, the cavity into which the device is placed is too large in diameter for the device. In some circumstances, the cavity wears either during placement of the screw or over time. In other circumstances back out involves rotation of the screw in the reverse direction to the rotation during introduction into an object. In some cases this problem has been solved by extruding cementitious or other fixing materials through a central cannula in the screw or fixation device which may allow evacuation of fixing materials at an end of the screw or at one or more ports along the side of the screw. This may have the benefit of securing the screw by limiting rotation and back out.

In surgical procedures, fixation devices are utilised to fix a plate or implant to a bone, to fix a bone to a bone, to provide compressive loading on a bone or for other purposes. In some cases, the fixation device is placed in bone and used to fix soft tissues back to bone. Generally a surgeon will position the bone or implant, then prepare a hole or access the bone with an awl through the bone or implant that is narrower than the diameter of the screw or fixation device being used. The screw or device can also have a self-cutting portion that does not require any prior preparation of the bone that the device is being placed in. The screw is then installed in the hole and rotated to provide compression and or fixation depending in the design of the device threads, length, head etc. A device can also be inserted by pushing into the bone and other means are used to fix the tissue or bone which may include sutures.

In these applications, polymeric bone cement, calcium phosphate cements, or other materials including meshes or fabrics as well as allograft can be used to assist in reducing the back out of the screw from its position or maintaining its position in an attempt to strengthen the construct in general; particularly in osteopenic bone. The use of a solid screw with a bone cement or other fillable material may increase the initial stiffness and strength of the repair, but may not significantly decrease loosening of the screw at the repair site. The substitution of solid screws with cannulated screws that can extrude a bone cement or other fillable material may improve the strength of the repair while at the same time reducing the likelihood that the screw will loosen and pull out, but distribution of the bone cement or fillable material through the screw and throughout the repair site remains a problem. The formation of a cement jacket around the bone screw following the implantation process increases the stability of the anchorage. This is of particular importance when the bone is of reduced quality, such as is the case for osteoporosis of the bony tissue for example.

In orthopedic or other medical or surgical applications, a cannula within the screw can be used to provide access to the implant/bone interface for delivery of materials other than bone cement such as biological actives, medications and other treatment materials.

### SUMMARY OF THE DISCLOSURE

The inventive concept may be applied to any device having a shaft for connecting objects or materials to one another.

Disclosed is an improved fixation device for facilitating mechanical fixation between two objects. The device is particularly described in terms of fixation for orthopaedic, spinal and surgical applications such as fixation between an implant and a bone region, between two bone regions or to place compression on fractured bone, fixation between a device and the bone that can be, but is not limited to, an interbody cage in the spine surgery , an acetabular cup in hip surgery, a tibial tray in knee surgery, a glenoid in shoulder surgery, the rotator cuff tendons in shoulder surgery, or any soft tissue that is connected back to bone. It may also be used in dental applications.

The device allows for access to the external surface of the fixation device at any point along the channel to deliver material or to provide line of sight access. It may also have the benefit of maintaining the strength of the fixation device while providing access for the placement of exogenous or endogenous materials or fluids such as cements, grouts, or other materials effecting fixation, or biologics or other actives to improve or influence biology.

In some forms, disclosed is a fixation device extending from a leading end to a trailing end, the device comprising a body comprising a shaft extending from the trailing end, an aperture being eccentric from a centre of the trailing end, and, a channel extending from the aperture and along at least a portion of an external surface of the body, the aperture and channel being aligned to form a passage.

In some forms, the channel and aperture are oriented to allow line of sight access to a location along the shaft that is distal the trailing end.

In some forms, disclosed is a securing element for association with a fixation device such as a cannulated screw. In some forms the securing element comprises at least one prong extending from a handle. In some forms the prong is adapted to be inserted into a cannulation in a screw.

In some forms the securing element comprises at least two prongs extending from a handle. In some forms the prongs are outwardly biased with respect to one another. In some forms the prongs vary in length or cross sectional properties when compared with one another.

The securing element may augment the fixation device which may allow the fixation device to resist rotation and therefore resist backout or pullout depending on the geometry of the securing element. The securing element provides control of rotation of the fixation device and allows for the securing element to be removed as needed. This can be critical in medical or non-medical fixation when back out can be detrimental but removal of a fixation device may be desired. The securing element may also provide additional pull-out resistance given the geometry of the elements as well as the spatial relationship to each other.

### BRIEF DESCRIPTION OF THE FIGURES

Embodiments will now be described by way of example only, with reference to the accompanying drawings in which:

FIG. 1 shows a side view of a fixation device of a first embodiment of the disclosure;

FIG. 2 shows a perspective view of the fixation device of FIG. 1;

FIG. 3 shows a head view of the fixation device of FIG. 1;

FIG. 4 shows a perspective view of a fixation device of a second embodiment of the disclosure;

FIG. 5 shows a tip perspective view of the fixation device of FIG. 4;

FIG. 6 shows a head perspective view of the fixation device of FIG. 4;

FIG. 7 shows a hidden lines view of the fixation device of FIG. 4;

FIG. 8 shows a perspective view of a fixation device of a third embodiment of the disclosure;

FIG. 9 shows a perspective view of an embodiment of the disclosure in use;

FIG. 10 shows a side view of an embodiment of a fixation device with cementitious material in use;

FIG. 11 shows a tip perspective view of the device of FIG. 10;

FIG. 12 shows a side view of a further embodiment of the disclosure;

FIG. 13 shows a trailing end view of the device of FIG. 12;

FIG. 14 shows a tip perspective view of the embodiment of FIG. 12

FIG. 15 shows a side view of a further embodiment of the disclosure;

FIG. 16 shows a perspective view of a further embodiment of the disclosure;

FIG. 17 shows a perspective view of a further embodiment of the disclosure;

FIG. 18 shows a side perspective view of the embodiment of FIG. 17;

FIG. 19 shows a perspective view of the embodiment of FIG. 17;

FIG. 20 shows a side view of a further embodiment of the disclosure;

FIG. 21 shows a hidden lines side view of the embodiment of FIG. 20;

FIG. 22 shows a trailing edge view of the embodiment of FIG. 20;

FIG. 23 shows a leading edge view of the embodiment of FIG. 20;

FIG. 24 shows a side perspective view of the embodiment of FIG. 20;

FIG. 25 shows a perspective head view of one embodiment of the disclosure;

FIG. 26 shows a perspective tip view of the embodiment of FIG. 25;

FIG. 27 shows a perspective head view of a securing element of one embodiment of the disclosure;

FIG. 28 shows a side view of a securing element of one embodiment of the disclosure;

FIG. 29 shows a tip view of the securing element of FIG. 28;

FIG. 30 shows a side view of the securing element of FIG. 28;

FIG. 31 shows a disassembled view of a fixation device and securing element of one embodiment of the disclosure;

FIG. 32 shows a cross sectional view of an assembled device of one embodiment of the disclosure;

FIG. 33 shows an assembled head view of the embodiment of FIG. 32;

FIG. 34 shows an assembled side view of the embodiment of FIG. 32;

FIG. 35 shows a side view of an assembled embodiment of another embodiment of the disclosure;

FIG. 36 shows a head view of the embodiment of FIG. 35;

FIG. 37 shows a side view of the embodiment of FIG. 35;

FIG. 38 shows a disassembled view of the embodiment of FIG. 35;

FIG. 39 shows a side view of one embodiment of the disclosure;

FIG. 40 shows a side view of the embodiment of FIG. 39;

FIG. 41 shows a head view of the embodiment of FIG. 39;

FIG. 42 shows a side perspective view of one embodiment of the disclosure;

FIG. 43 shows a head view of the embodiment of FIG. 42;

FIG. 44 shows a disassembled perspective view of the embodiment of FIG. 42;

FIG. 45 shoes a side view of a securing element of one embodiment of the disclosure;

FIG. 46 shows a side perspective view of a fixation device of one embodiment of the disclosure;

FIG. 47 shows a cross-sectional assembled view of the embodiment of FIGS. 45 and 46;

FIG. 48 shows an assembled view of the embodiment of FIGS. 45 and 46.

### DETAILED DESCRIPTION OF EMBODIMENTS

In the following detailed description, reference is made to the accompanying drawings which form a part of the detailed description.

Disclosed broadly is a fixation device such as a screw or plug for fixing objects to one another. The fixation device comprises a shaft extending from a leading end which is designed to be inserted into an object to engage the object and a trailing end which is designed to be located external to or at an edge of the object when the device is in use fixing objects to one another. In some forms the trailing end includes a head that has a wider diameter than the shaft. In some forms the device includes an external helical thread. In some forms, disclosed is a fixation device extending from a leading end to a trailing end, the device comprising a body comprising a shaft extending from the trailing end, an aperture being eccentric from a centre of the trailing end, and, a channel extending from the aperture and along at least a portion of an external surface of the body, the aperture and channel being aligned to form a passage.

In some forms, the channel and aperture are oriented to allow line of sight access to a location along the shaft that is distal the trailing end.

In some forms, the fixation device includes a head and the aperture extends through the head. In some forms, the aperture is open at one side.

In some forms, the shaft includes a helical thread along at least a portion of the shaft.

In some forms, the channel extends through a threaded region of the shaft such that the helical thread includes cut-outs in line with the channel.

In some forms, the channel is at an angle to the longitudinal axis of the fixation device. In some forms the channel is an open channel.

In some forms the device includes a plurality of passages.

In some forms the cut-outs are shaped to effect accumulation of the material inside the passage(s) or cut-outs.

In some forms the exterior surface of the fixation device is modified or augmented by insertion of material after insertion of the fixation device.

In some forms, disclosed is a fixation device comprising a head, a body comprising a shaft extending from the head, the shaft including a helical thread along at least a portion of the shaft, an aperture extending through the head, the aperture being eccentric from a centre of the head, and, a channel extending from the aperture and along at least a portion of an external surface of the body, the aperture and channel being aligned to form a passage. In some forms, disclosed is a fixation device that is a headless, a body comprising a shaft extending from the head, the shaft including a helical thread along at least a portion of the shaft, an aperture extending through the head, the aperture being eccentric from a centre of the head, and, a channel extending from the aperture and along at least a portion of an external surface of the body, the aperture and channel being aligned to form a passage. Also in some forms, disclosed is a device such as a locking screw comprising a head that is threaded and locked to another device such as a nail or a plate.

Generally the application discloses a fixation device that includes an eccentric passage extending through the fixation device from the trailing end of the fixation device and along an external surface of the shaft of the fixation device.

The fixation device is suitable for all fixation applications but is being described with reference to orthopedic references in particular, that is such applications as arthroplasty device fixation, pedicle screws in bone, spinal devices and fusion devices in bone, trauma devices and systems, standalone screws. The fixation device is also suitable for screw or plug devices that are placed into bone and then soft tissues are re-attached to using suture that is connected to the device.

The fixation device has the benefit of allowing a user to access the interface between the device and a bone or other material or the interface between an implant and the bone after the fixation device is positioned to fix one object to another. The external channel allows for delivery of material such as cementitious material or medication to a site specific location along the channel. It also allows for line of sight access from the head or top of the device down the body of the fixation device to allow for accurate and precise positioning of material. This allows for a reduction in delivery quantity because the delivery is to a specific, targeted, site.

In some forms the channel or channels further allow delivery of more than one substance to specific sites. For example, multiple biologics or a cement and an activator could be delivered to adjacent sites to allow interaction.

The strength of a fixation device such as a screw is dependent upon the smallest diameter of the device, which may be a core or a shaft diameter. In some forms the external channel does not reduce the smallest diameter in the screw or weaken the shaft strength.

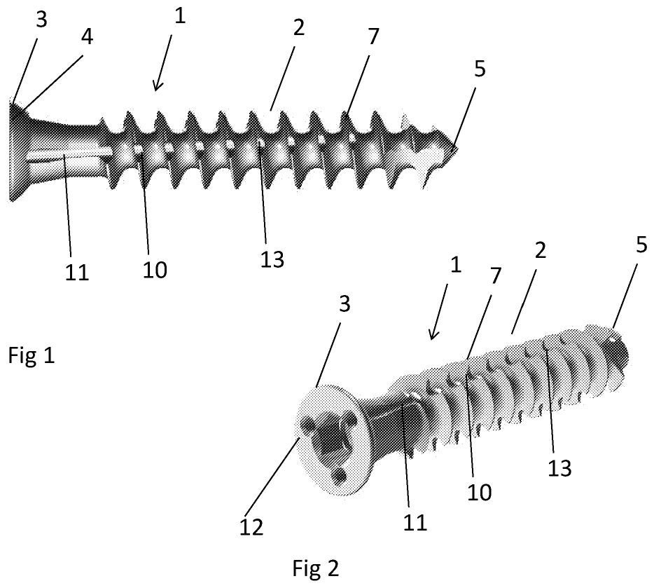

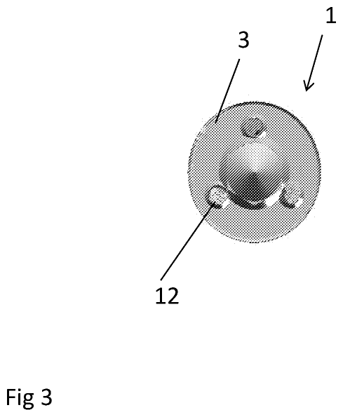

Referring now to FIGS. 1-3, disclosed is a fixation device 1 in the form of a screw having a shaft 2 extending from a trailing end having a head 3. The head 3 has a greater diameter than the shaft 2. In the illustrated form the head has a rounded shaft end 4. The shaft 2 comprises an elongate body extending from the head 3 to a tip 5. The shaft includes a thread 7 extending outwardly in a helical spiral about the shaft 3. In the illustrated form the thread 7 extends outwardly from a substantial portion of the shaft, however the thread may be any length.

The fixation device 1 further includes a passage 10 comprising a channel 11 which extends along a portion of an external surface of the shaft 2 and an aperture 12 which extends through the head 3. The passage 10 is oriented and sized to allow access to a contact surface between the fixation device and the material the fixation device is fixing. This access allows a user to have a line of sight to a position distal the head when the fixation device is positioned within an object or bone. Further, the access allows material such as cementitious material to be delivered to a site specific position distal the head.

The aperture 12 is positioned eccentrically with respect to a centre of the head. The aperture 12 may be offset to a position on the head 3 that is fully or partly unaligned with the shaft 2. That is, the aperture extends through the head 3 to a position that is at least partly external to the shaft of the fixation device. In the illustrated form the apertures 12 are located toward a periphery of the head 3 such that they do not interfere with an actuator in the form of a socket or screw head mechanism.

The aperture 12 and the channel 11 are positioned to align with one another such that the aperture 12 and channel 11 form a passage 10 extending from the head to a point distal the head on the shaft. This allows a user to access site specific positions along the channel from a location outside the material the fixation device is positioned in through the aperture 12 in the head 3.

In the illustrated form, three apertures and three corresponding channels are spaced apart about the head and shaft. It will be clear that more or fewer passages fall within the scope of the disclosure.

The channel 11 is positioned to extend along at least a portion of the surface of the shaft 2 from the head to a position distal the head. The channel in the illustrated form is unenclosed, having an open cross section as it extends along the shaft 2. The channel breaks the thread as it meets the thread, forming cut-outs 13 of the thread that form part of the channel.

The passages 10 are generally angled such that a line of sight is possible when the fixation device is positioned within material. The angle must be small enough to continue the required distance along the surface of the fixation device.

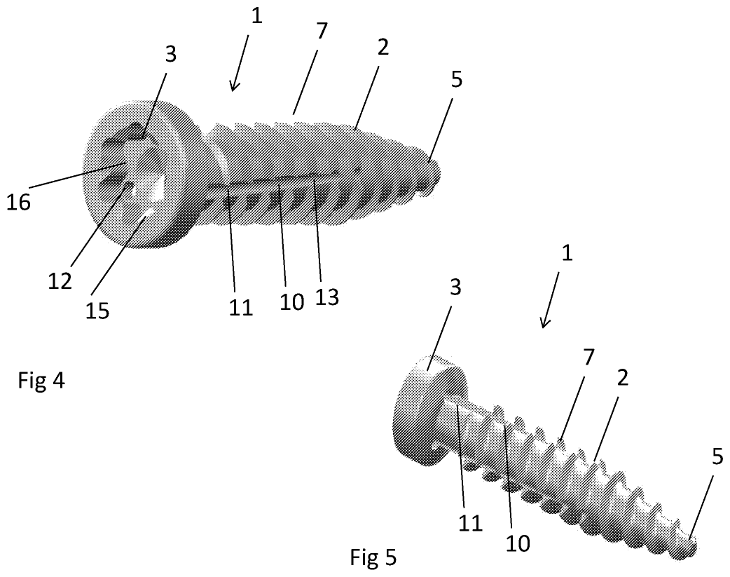

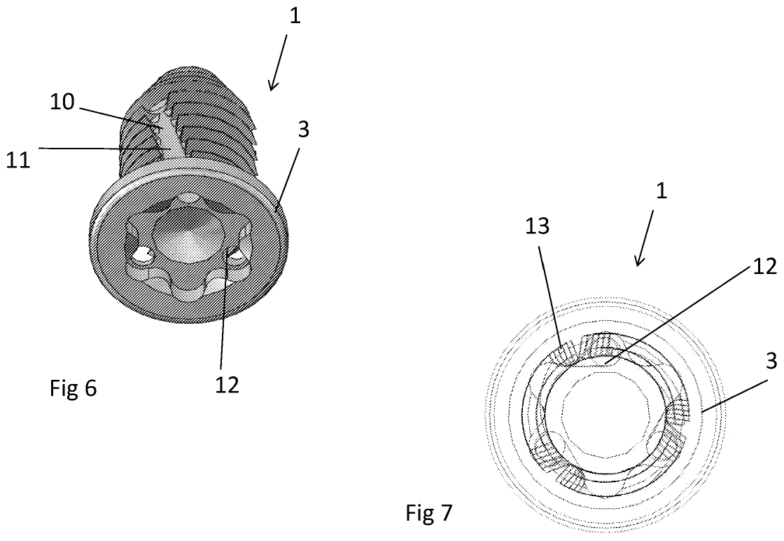

Referring now to FIGS. 4 to 7, disclosed is a fixation device 1 in the form of a screw having a shaft 2 extending from a head 3. The head 3 includes a profile 15 in its upper surface that allows for rotation of the fixation device 1. In this illustrated form, the apertures 12 are positioned within the depression 15 created by the profile 15 in the head. The apertures 12 extend through the head to connect with a corresponding channel 11 extending along a portion of the external surface of the shaft and forming cut outs in a thread extending outwardly from the shaft.

As shown best in FIG. 7, the helical nature of the thread 7 and the angled nature of the channel 11 means the cut-outs 13 are positioned offset with respect to one another as the channel proceeds down the shaft. That is, the position of the cut-outs is arranged such that the cut-outs are misaligned in a longitudinal direction but aligned along the angle of the passage.

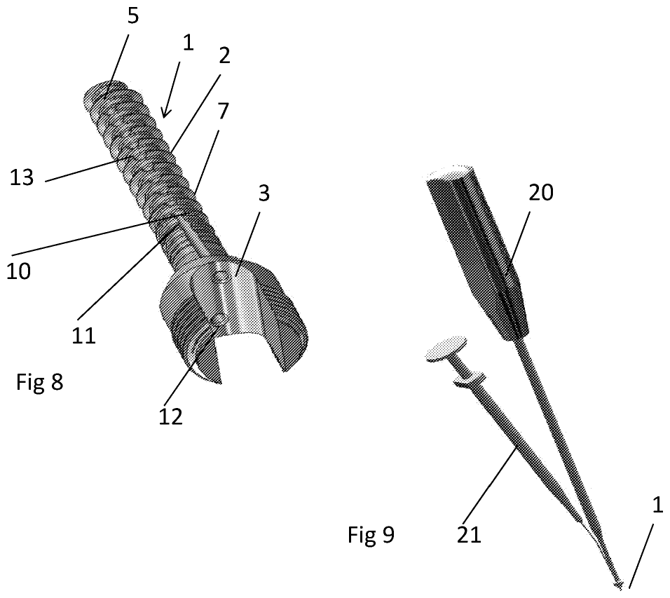

Referring to FIG. 8, in some forms the fixation device may be a pedicle screw or other alternative screw shapes with the apertures 12 and channel 11 positioned for best fit to the screw head 3. In this form two passages are shown extending from either side of the head.

Referring now to FIG. 9, in use a screwdriver 20 or other rotator is utilised to position the fixation device. An injector 21 such as a syringe can be utilised to deliver material such as bone cement to a site specific location along the length of the channel on the screw.

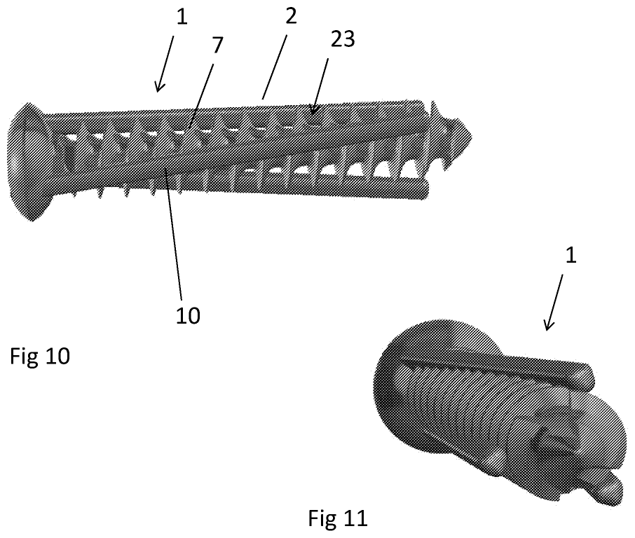

Referring now to FIGS. 10 and 11, injection of cementitious material into the channels may in some forms create a multi-modal locking mechanism 23 in the passages 10 that reduces backout and pullout capacity for the fixation device.

In use in a cortical bone application, the fixation device is inserted into cortical bone. In some forms the channel is then cleared out using, for example, a wire. Then cementitious or other material is inserted into the channel along a line of sight on the helical screw. In some forms the cementitious material extends along the channels to reduce back out.

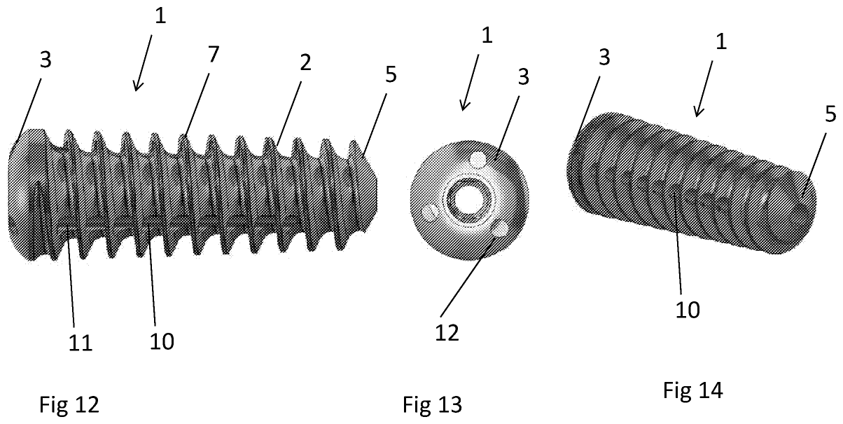

Referring now to FIGS. 12 through 14, disclosed is a fixation device 1 comprising a shaft 2 extending from a trailing end 3. In this form the trailing end 3 does not include a head. The shaft 2 comprises an elongate body extending from the trailing end 3 to a tip 5. The shaft includes a thread 7 extending outwardly in a helical spiral about the shaft 3. In the illustrated form the thread 7 extends outwardly from a substantial portion of the shaft, however the thread may be any length,

The fixation device 1 further includes a passage 10 comprising a channel 11 which extends along a portion of an external surface of the shaft 2 and an aperture 12 which extends through the trailing end 3. The passage 10 is oriented and sized to allow access to a contact surface between the fixation device and the material the fixation device is fixing. This access allows a user to have a line of sight to a position distal the head when the fixation device is positioned within an object or bone. Further, the access allows material such as cementitious material to be delivered to a site specific position distal the head.

The aperture 12 is positioned eccentrically with respect to a centre of the trailing end 3. The aperture 12 may be offset to a position on the trailing end 3 that is fully or partly unaligned with the shaft 2. That is, the aperture extends through the head 3 to a position that is at least partly external to the shaft of the fixation device. In the illustrated form the apertures 12 are located toward a periphery of the trailing end 3 such that they do not interfere with an actuator in the form of a socket or screw head mechanism.

The aperture 12 and the channel 11 are positioned to align with one another such that the aperture 12 and channel 11 form a passage 10 extending from the trailing end to a point distal the trailing end and proximal the tip on the shaft. This allows a user to access site specific positions along the channel from a location outside the material the fixation device is positioned in through the aperture 12 in the head 3.

In the illustrated form, three apertures and three corresponding channels are spaced apart about the head and shaft. It will be clear that a larger or smaller number of passages fall within the scope of the disclosure.

The channel 11 is positioned to extend along at least a portion of the surface of the shaft 2 from the trailing end to a position distal the trailing end. In some forms the channel generally extends substantially in line with the surface of the shaft 2. The channel in the illustrated form is enclosed when extending through the helical thread but unenclosed when not extending through the helical thread, having an open cross section as it extends along the shaft 2. The channel breaks the thread as it meets the thread, forming cut-outs 13 of the thread that form part of the channel.

The passages 10 are generally angled such that a line of sight is possible when the fixation device is positioned within material. The angle must be small enough to continue the required distance along the surface of the fixation device. This allows a user at the trailing end to have line of sigh access to a point distal the trailing end.

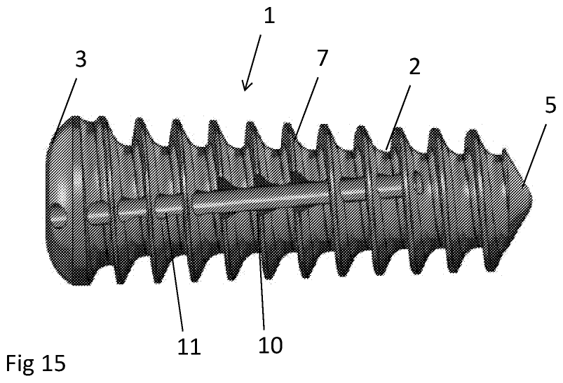

Referring now to FIG. 15, in some forms the channel is enclosed by the helical thread at some points along the shaft but is open at other points along the shaft.

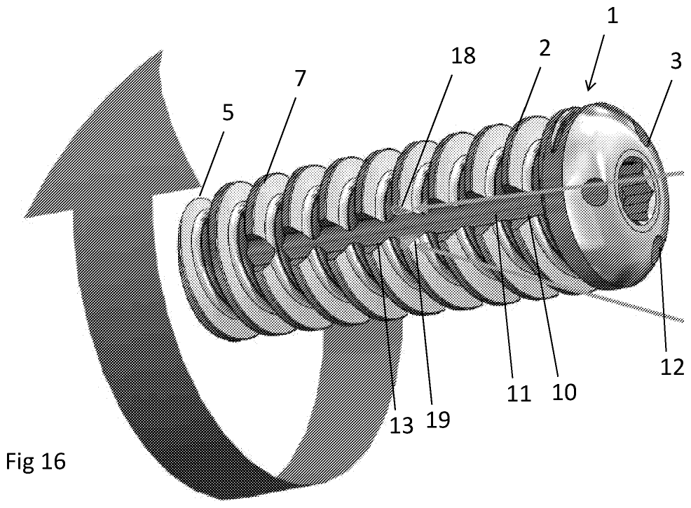

As shown in FIG. 16, the shape of the cut-outs 13 can be utilised to effect a shoulder to allow ingrowth or ongrowth and fixation to the bone. This will allow the fixation device to resist axial load and will reduce torsional back out of the fixation device. In this form the device rotates in the direction of the arrow and the cut-outs 13 in the thread are shaped to have a first end 18 and a second end 19 along the helical thread, the cut-outs 13 oriented such that the first end 18 of the cut-out extends a smaller distance from the shaft than does the second end 19 of the cut-out so that on rotation of the device, when the second end 19 trails the first end, the second end acts to push material into the cut-out and therefore into the channel. The first end which acts as the leading end can be profiled to best allow material to move over the first end while the second end which acts to trail the first end on rotation can be profiled for greatest scooping or scraping effect to capture material in the channel. This results in a “bulldozer” action as the screw advances into the bone. This action is improved by using a fully tapered shaft and thread.

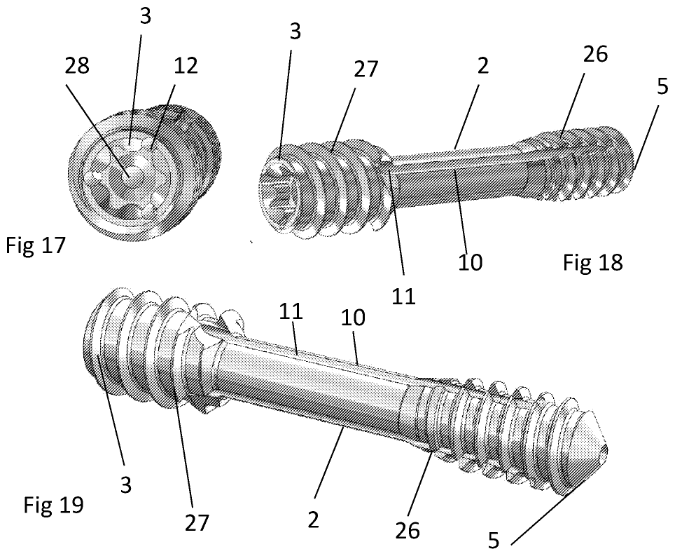

Referring now to FIGS. 17 through 19, disclosed in some forms is a fixation device 1 in the form of a screw. In this embodiment the screw is a Herbert style screw. The device 1 comprises a shaft 2 extending from a trailing end 3. A leading thread section 26 and a trailing head section 27 extend outwardly from the shaft 2 at either end of the device 1. The trailing end 3 includes apertures 12 extending there through in a longitudinal direction. The apertures 12 are aligned with a channel 11 which extends along the surface of the shaft 2. The apertures 12 and respective channels 11 form passages 10. The passages 10 provide line of sight access to the leading thread section 26 from the trailing end 3. The length of the screw means the passages 10 are not angled as they are in other embodiments. The device 1 further includes a central cannula 28 extending from the trailing end toward the tip 5.

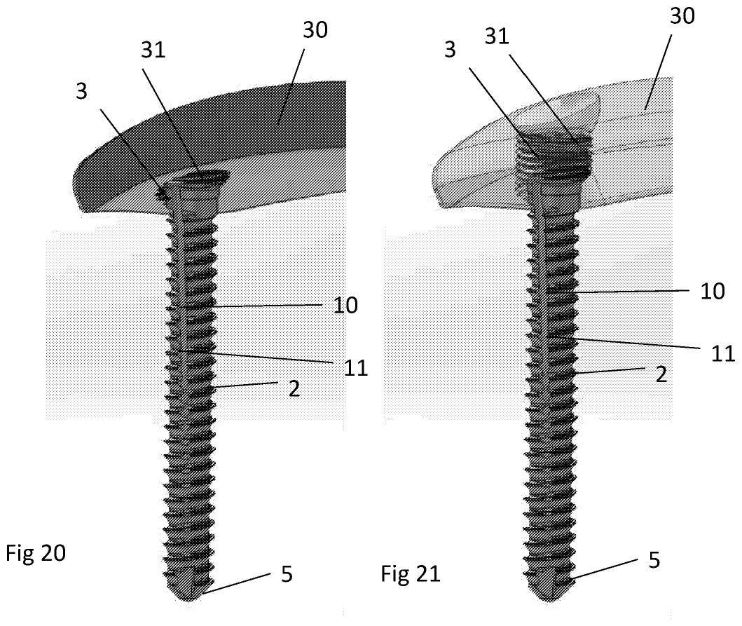

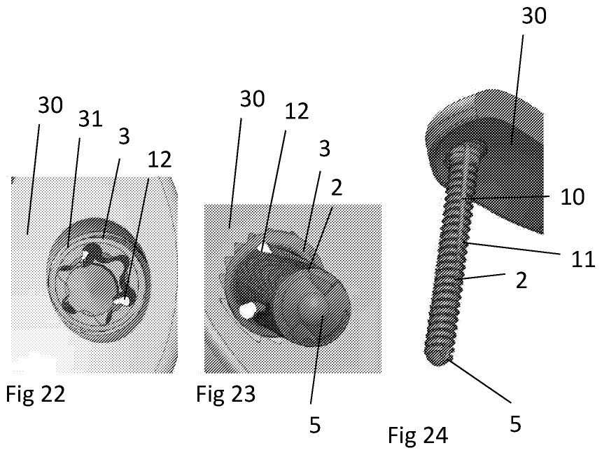

Referring now to FIGS. 20 to 24, disclosed is a fixing device in the form of a locking screw. Locking screws may be locked to a bone plate or other device such as a Nail. In the illustrated form the device 1 is locked into the bone plate 30 through a plate aperture 31 through use of a threaded head 3 that is located at the trailing end of the device. The threaded head 3 can be accessed through the plate aperture 31 in the bone plate 30. The device includes apertures 12 which extend through the threaded head 3 to align with the channels 11. The apertures 12 and channels 11 form a passage 10 along the outer surface of the locking screw. These passages 10 can be accessed from a location beyond the bone plate through the plate aperture 31 to give line of sight access to the leading end of the device from beyond the bone plate.

In some not illustrated forms, the eccentric passage may be offset from the center of the head only slightly, to allow the passage to continue through a portion of the shaft and extend through the side of the shaft. Alternatively in other not illustrated forms the eccentric passage may be substantially offset from the center of the head such that the passage breaks the threaded surface of the shaft but does not extend through the body of the shaft.

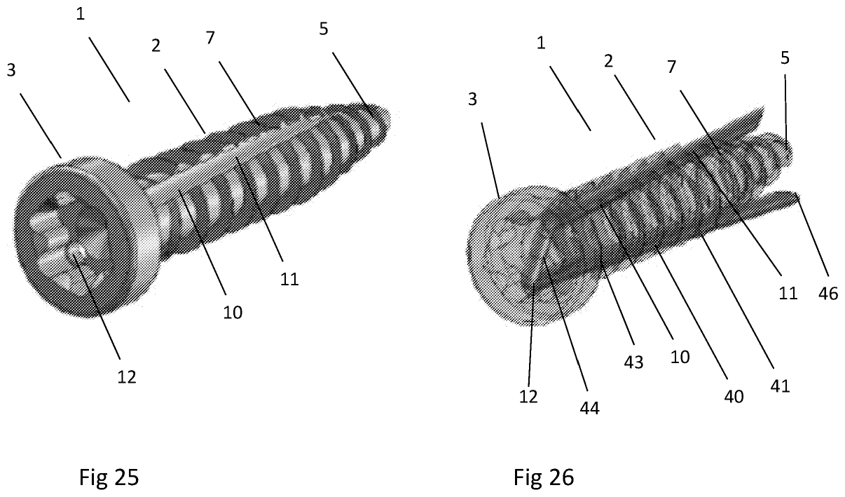

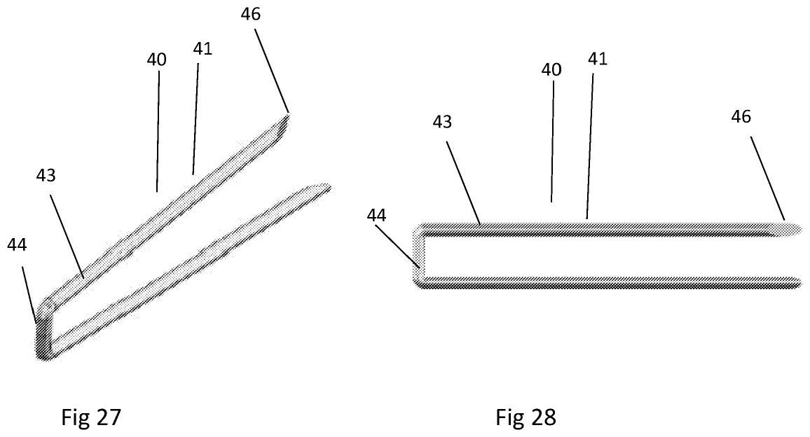

Referring now to FIGS. 25-27, disclosed is a fixation device 1 in the form of a screw having a shaft 2 extending from a trailing end having a head 3. The head 3 has a greater diameter than the shaft 2. The shaft 2 comprises an elongate body extending from the head 3 to a tip 5. The shaft includes a thread 7 extending outwardly in a helical spiral about the shaft 3. In the illustrated form the thread 7 extends outwardly along the length of a substantial portion of the shaft, however the thread may be any length.

The illustrated fixation device 1, further includes a passage 10 comprising two channels 11 which extend along a portion of an external surface of the shaft 2. Each channel 11 extends from an aperture 12 which extends through the head 3. The passage 10 is oriented and sized to allow access to a contact surface between the fixation device and the material the fixation device k fixing.

The aperture is positioned eccentrically with respect to a centre of the head. The aperture may be offset to a position on the head 3 that is fully or partly unaligned with the shaft 2. In the illustrated form the apertures are located toward a periphery of the head 3 such that they do not interfere with an actuator in the form of a socket or screw head mechanism.

The aperture and the channel 11 are positioned to align with one another such that the aperture and channel 11 form the passage 10 extending from the head to a point distal the head on the shaft. This allows a user to access site specific positions along the channel from a location outside the material the fixation device is positioned in through the aperture in the head 3.

The channel 11 is positioned to extend along at least a portion of the surface of the shaft 2 from the head to a position distal the head. The channel in the illustrated form is unenclosed, having an open cross section as it extends along the shaft 2. The channel breaks the thread as it meets the thread, forming cut-outs 13 of the thread that form part of the channel.

The passages 10 are generally angled such that a line of sight is possible when the fixation device is positioned within material. The angle must be small enough to continue the required distance along the surface of the fixation device. The passages may be parallel to a centre axis of the fixation device 1. Alternatively the passages may be angled with respect to the centre axis of the fixation device and may be angled with respect to one another.

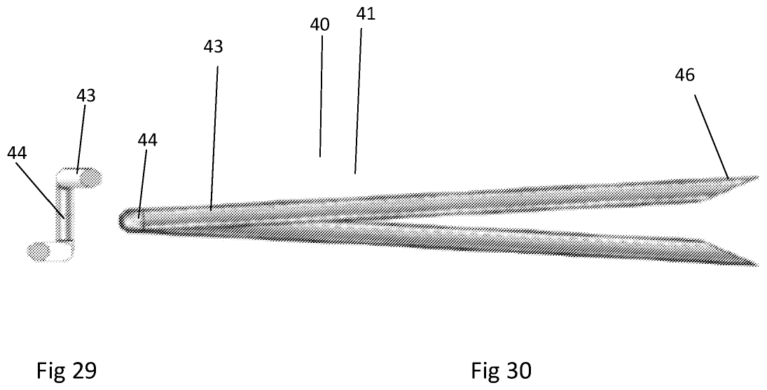

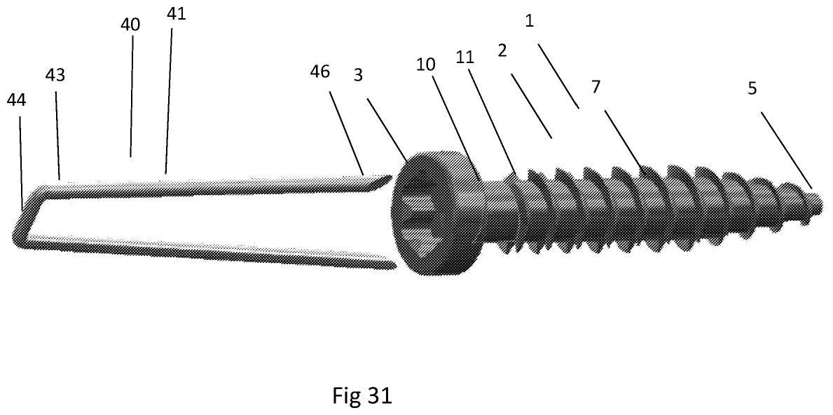

A rotational securing element 40 in the form of a staple 41 is shown best in FIGS. 28-30 and is adapted to be associated with the fixation device 1. The rotational securing element 40 in this illustrated form comprises two prongs 43 or stems extending from a handle in the form of a bar 44. In some forms the handle is configured to be located outside the passages in the fixation device. Each prong 43 is biased outwardly with respect to one another as it extends away from the bar 44. In the illustrated form the prongs 43 extend outwardly with respect to one another in two dimensions.

Referring to FIG. 31, in use, after insertion of the fixation device 1 into an object to be secured, the rotational securing element 40 is inserted into the two passages 10 in the fixation device 1. In order to insert the securing element into the passages, the prongs 43 may be pushed into alignment with the passages. Once the prongs are inserted into the passages, the shape of the passages and the apertures will guide movement of the prongs in insertion. The prongs are biased back into their outwardly extending position and therefore are biased into a locked or secured position within the fixation device.

The prongs 43 of the illustrated form have a tapered end 46 to allow for movement of the prongs through the passages without catching against the threads or the fixation device. In some forms the tapered tip is tapered symmetrically, in some forms the tip is pointed, in some forms the tip is asymmetrically tapered.

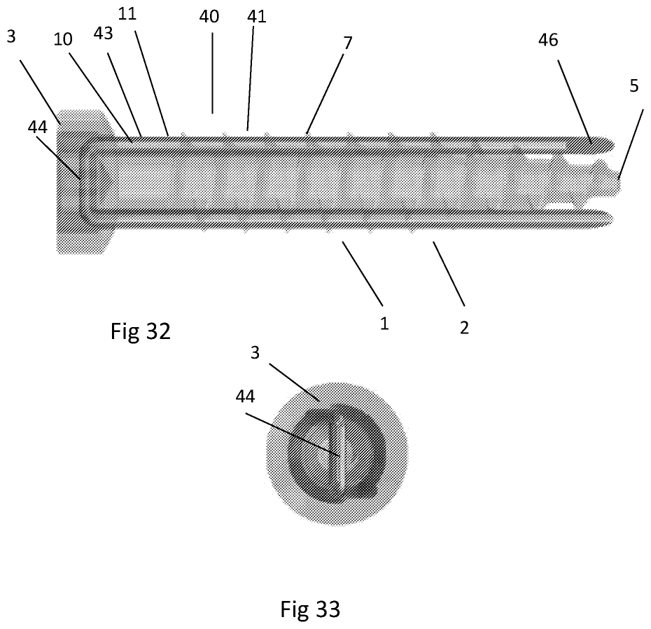

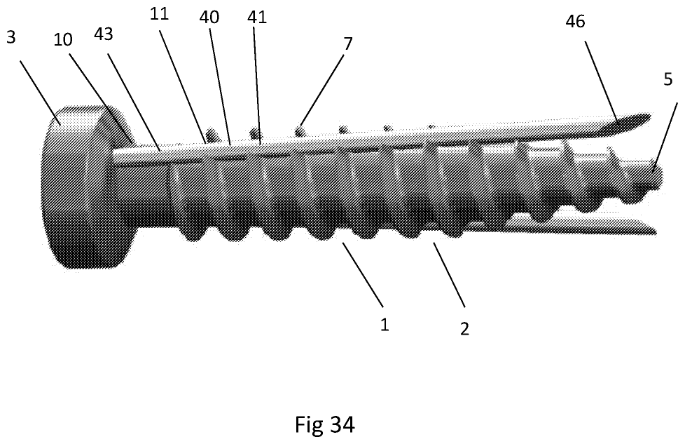

As shown in FIGS. 32-34, once the prongs are inserted into the passages such that the bar 44 abuts the head 3 of the fixation device 1, the securing element is in place. The positioning of the securing device resists back-out by the fixation device 1 in that rotation of the fixation device is limited by the presence of the prongs 43.

Removal of the securing device is possible without damaging the fixation device 1, the securing device 40 or the object in which the fixation device is inserted. Removal in some forms requires force and in some forms can only be performed with specialised tools such as, for example, pliers. The shape, configuration and bias of the securing element 40 resists walk-out of the securing element from the passages.

The securing device allows he insertion torque to be significantly less than the removal torque.

The material of the securing element 40 may comprise any material that has the flexibility to be biased into the configuration desired. For example, the material may comprise titanium, cobalt, chrome, nitinol or stainless steel. In some forms the securing element may be resorbable, hydrophobic, hydrophilic, metal, plastic.

In some forms the rotational securing element is formed with any cross section. It may have a round, square, triangular, semicircular, T or I cross section, tear shaped, blade shaped or angular. Moreover, the surface of the securing element may be textured, smooth, serrated, coated or have any other surface. In some forms the securing element is formed of wire or filaments.

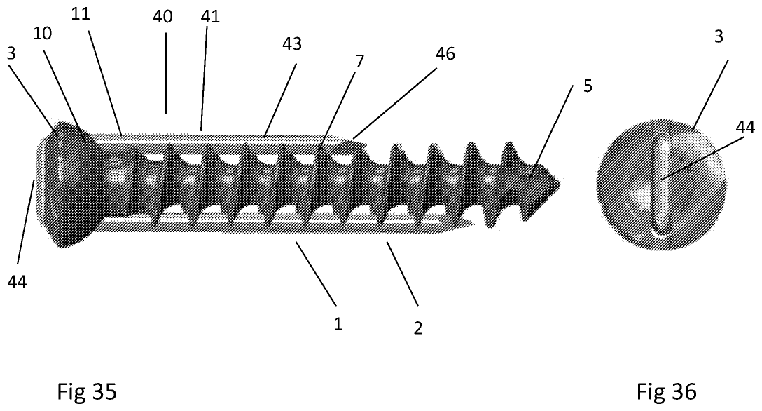

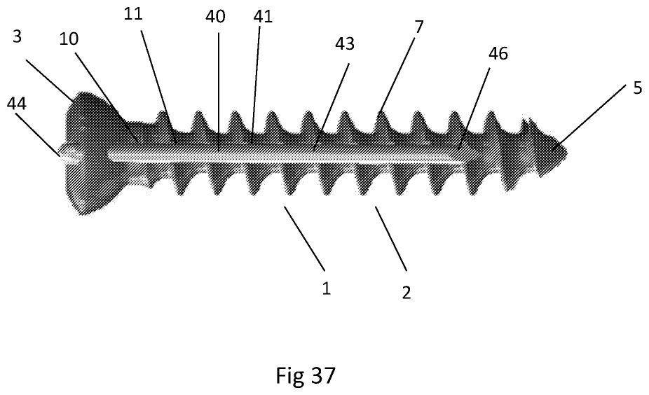

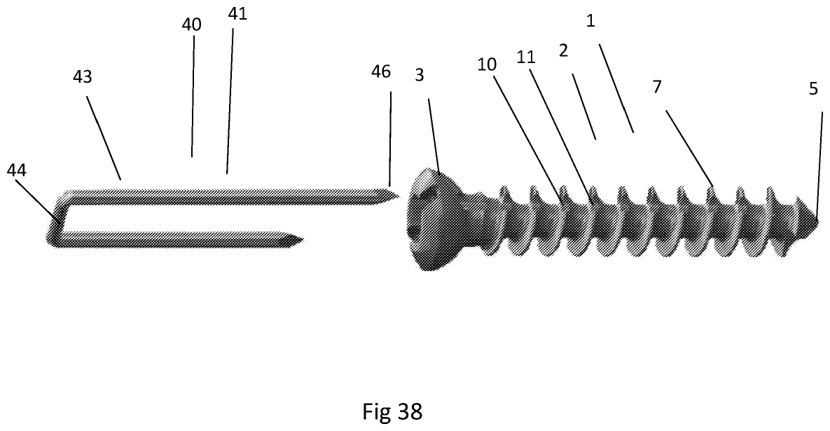

In some forms, as shown in FIGS. 35-38, the securing element 40 has two prongs of different lengths. In some forms the prongs are parallel to one another and are not biased outwardly with respect to one another.

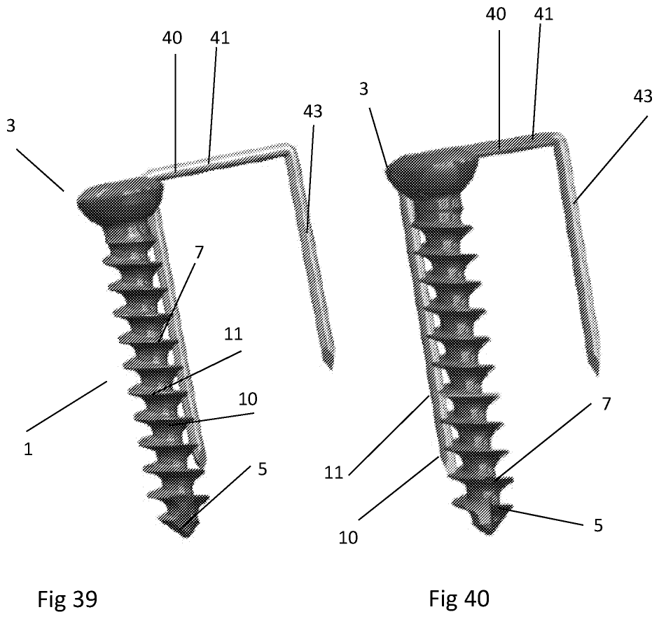

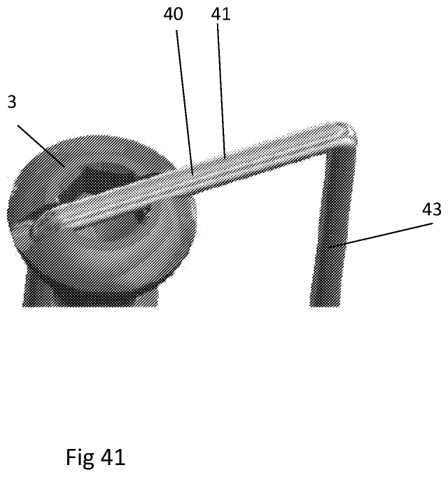

In some forms as best shown in FIGS. 39-41, the securing element comprises two prongs 43 and 43A and is adapted to engage the screw with one prong 43 and to engage surrounding object or tissue with the second prong 43A. In this form the securing element 40 provides two functions, resisting back-out of the screw through provision of a longitudinal shoulder and capturing and stabilising the object or tissue in which the fixation device is inserted.

As shown in FIGS. 39 and 40, the orientation of the securing element may be over the head 3 of the fixation device 1, or may be away from the fixation device. The orientation may also be anywhere in between these orientations.

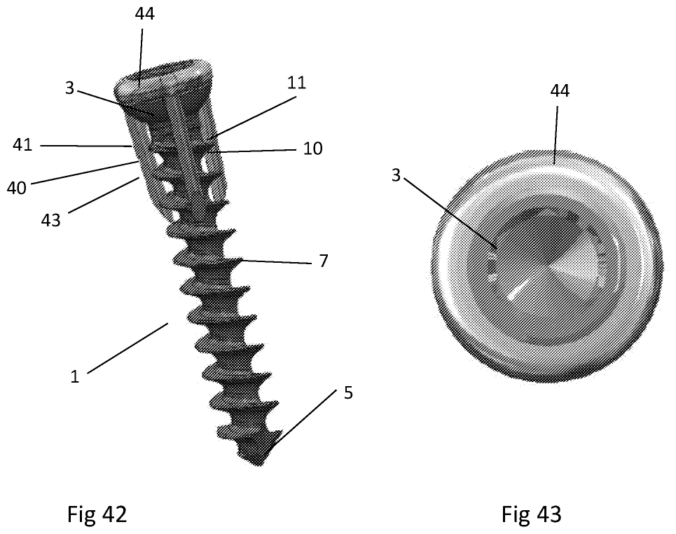

Referring now to FIGS. 42-44, disclosed is a fixation device 1 in the form of a screw having a shaft 2 extending from a trailing end having a head 3. The head 3 has a greater diameter than the shaft 2. The shaft 2 comprises an elongate body extending from the head 3 to a tip 5. The shaft includes a thread 7 extending outwardly in a helical spiral about the shaft 3. In the illustrated form the thread 7 extends outwardly along the length of a substantial portion of the shaft, however the thread may be any length.

The illustrated fixation device 1 further includes a passage 10 comprising three channels 11 which extend along a portion of an external surface of the shaft 2. In this illustrated form the channels extend along approximately a third of the shaft. Each channel 11 extends from an aperture 12 through the head 3. The passage 10 is oriented and sized to allow access to a contact surface between the fixation device and the material the fixation device is fixing.

The apertures 12 are positioned eccentrically with respect to a centre of the head. The apertures may be offset to a position on the head 3 that is fully or partly unaligned with the shaft 2. In the illustrated form the apertures are located at a periphery of the head 3 such that they do not interfere with an actuator in the form of a socket or screw head mechanism and form a partial unenclosed circle or cutout.

Each aperture and respective channel 11 are positioned to align with one another such that the aperture and channel 11 form the passage 10 extending from the head to a point distal the head on the shaft.

The channels 11 are positioned to extend along at least a portion of the surface of the shaft 2 from the head to a position distal the head. The channel in the illustrated form is unenclosed, having an open cross section as it extends along the shaft 2. The channel breaks the thread as it meets the thread, forming cut-outs :13 of the thread that form part of the channel.

The passages 10 are generally angled such that a line of sight is possible when the fixation device is positioned within material. The angle must be small enough to continue the required distance along the surface of the fixation device. The passages may be parallel to a centre axis of the fixation device 1. Alternatively, the passages may be angled with respect to the centre axis of the fixation device and may be angled with respect to one another.

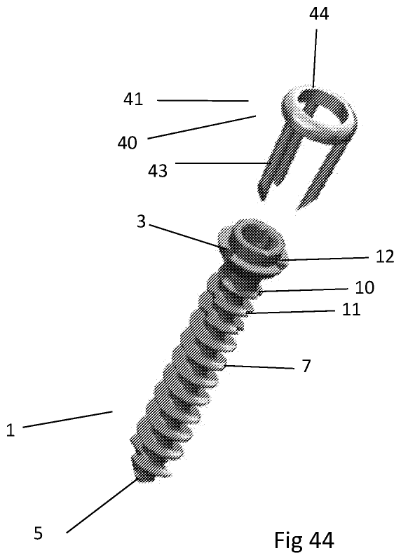

A rotational securing element 40 in the form of a cap 41 is shown best in FIG. 44 and is adapted to be associated with the fixation device 1. The rotational securing element 40 in this illustrated form comprises three prongs 43 or stems extending from a handle in the form of a ring 44. Each prong 43 in the illustrated form extends parallel to one another and to a centre axis of the fixation device as it extends away from the ring 44. In non-illustrated forms the prongs 43 extend outwardly with respect to one another.

Referring to FIG. 42, in use, after insertion of the fixation device 1 into an object to be secured, the rotational securing element 40 is inserted into the three passages 10 in the fixation device 1.

The prongs 43 of the illustrated form have a tapered end 46 to allow for movement of the prongs through the passages without catching against the threads or the fixation device. In some forms the tapered tip is tapered symmetrically, in some forms the tip is pointed, in some forms the tip is asymmetrically tapered.

As shown in FIG. 42, once the prongs are inserted into the passages such that the ring 44 abuts the head 3 of the fixation device 1, the securing element is in place. The positioning of the securing device resists back-out by the fixation device 1 in that rotation of the fixation device is limited by the presence of the prongs 43,

Removal of the securing device is possible without damaging the fixation device 1, the securing device 40 or the object in which the fixation device is inserted. Removal in some forms requires force and, in some forms, can only be performed with specialised tools such as, for example, pliers.

In some forms the rotational securing element formed with any cross section. It may have a round, square, triangular, semicircular, T or I cross section. Moreover, the surface of the securing element may be textured, smooth, serrated, coated or have any other surface.

In some forms the prongs of this embodiment may extend outwardly. In other forms the prongs may be curved. In use the prongs 43 are deformed to straight or parallel. Once the securing element 40 is inserted the prongs 43 are released and act to resist migration of the securing element.

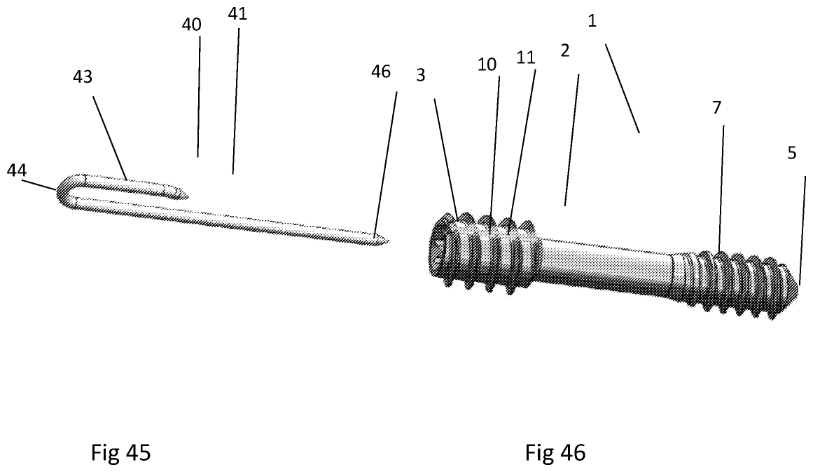

Referring now to FIGS. 45-48, in some forms disclosed is a fixation device 1 in the form of a screw having a shaft 2 extending from a trailing end having a head 3. The shaft 2 comprises an elongate body extending from the head 3 to a tip 5. The shaft includes a thread 7 extending outwardly in a helical spiral about the shaft 3. In the illustrated form the thread 7 extends outwardly along two portions of the shaft, however the thread may be any length.

The illustrated fixation device 1 further includes a central cannulation 47 and a passage 48 which extends along a portion of an external surface of the shaft 2. The passage 48 is oriented and sized to allow access to a contact surface between the fixation device and the material the fixation device is fixing.

A rotational securing element 40 in the form of a staple 41 is shown best in FIG. 45 and is adapted to be associated with the fixation device 1. The rotational securing element 40 in this illustrated form comprises two prongs 43 and 43A or stems extending from a handle in the form of a bar 44. In the illustrated form the prongs 43 and 43A are different in length with the shorter prong including a hooked or curved end 49.

In use, after insertion of the fixation device 1 into an object to be secured, the rotational securing element 40 is inserted into the two passages 10 in the fixation device 1.

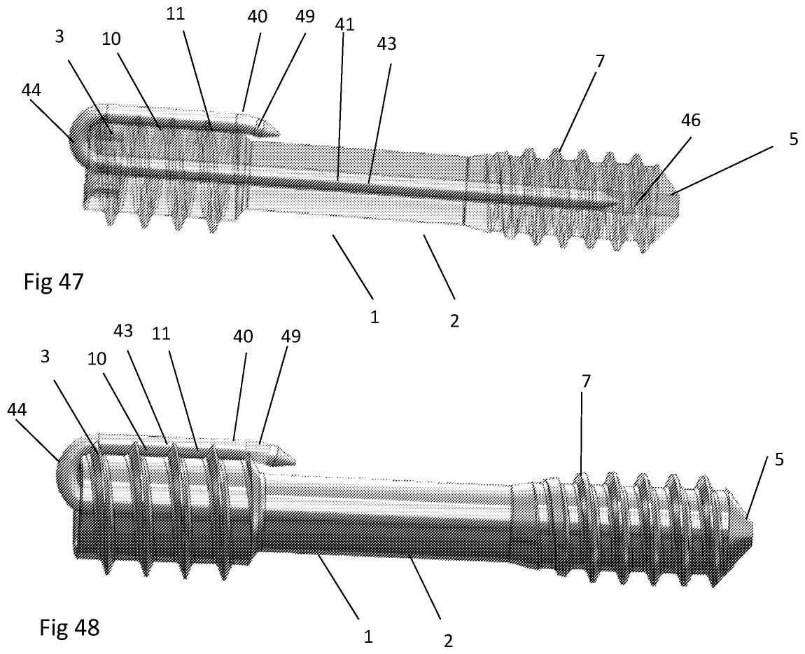

As shown in FIG. 47, once the prongs are inserted into the passages such that the bar 44 abuts the head 3 of the fixation device 1, the securing element is in place. The hooked end 49 locks over or clips onto a shoulder or projection on the fixation device. The positioning of the securing device resists back-out by the fixation device 1 in that rotation of the fixation device is limited by the presence of the prongs 43.

Removal of the securing device is possible without damaging the fixation device 1, the securing device 40 or the object in which the fixation device is inserted. Removal in some forms requires force and in some forms can only be performed with specialised took such as, for example, pliers. The hooked or curved end 49 resists walk-out of the securing element from the passages.

In some forms the rotational securing element is formed with any cross section. It may have a round, square, triangular, semicircular, T or I cross section. Moreover the surface of the securing element may be textured, smooth, serrated, coated or have any other surface. In some forms the securing element is formed of wire or filaments.

The use of a securing element to augment a fixation device and resist rotation and therefor backout provides control of rotation and allows for the securing element to be removed as needed. This can be critical in medical or non-medical fixation when removal of a fixation device may be desired

It will be clear to a person skilled in the art that there are other embodiments that will fall under the disclosure.

While the device has been described in reference to its preferred embodiments, it is to be understood that the words which have been used are words of description rather than limitation and that changes may be made without departing from the scope of the application as defined by the appended claims.

It is to be understood that a reference herein to a prior art document does not constitute an admission that the document forms part of the common general knowledge in the art in Australia or in any other country.

In the claims which follow and in the preceding description, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the device of the disclosure.

1. A fixation device extending from a leading end to a trailing end, the device comprising:
a shaft extending from the trailing end,
an aperture extending through the trailing end being eccentric from a centre of the trailing end, and,
a channel extending from the aperture and along at least a portion of an external surface of the shaft, the aperture and channel being aligned to form a passage.

2. A fixation device as defined in claim 1, wherein the channel and aperture are oriented to allow line of sight access to a location along the shaft that is distal the trailing end.

3. A fixation device as defined in claim 1 or 2, wherein the fixation device includes a head and the aperture extends through the head.

4. A fixation device as defined in any one of the preceding claims, wherein the aperture is open at one side.

5. A fixation device as defined in any one of the preceding claims wherein the shaft includes a helical thread along at least a portion of the shaft.

6. A fixation device as defined in claim 5, wherein the channel extends through a threaded region of the shaft such that the helical thread includes cut-outs in line with and defining the channel.

7. A fixation device as defined in claim 6, wherein the cut-outs in the thread are shaped to have a first end and a second end along the helical thread, the cut-outs oriented such that the first end of the cut-out extends a smaller distance from the shaft than does the second end of the cut-out so that on rotation of the device, when the second end trails the first end the second end acts to push material into the cut-out.

8. A fixation device as defined in any one of the preceding claims, wherein the channel is at an angle to the longitudinal axis of the fixation device.

9. A fixation device as defined in any one of the preceding claims, comprising a plurality of passages.

10. A fixation device as defined in any one of the preceding claims, wherein the channel is an open channel.

11. A securing element for association with a fixation device having a cannulation, the securing element comprising at least one prong extending from a handle, the prong being adapted to be inserted into the cannulation and the handle adapted to remain external to the cannulation.

12. A securing element as defined in claim 10, comprising at least two prongs, the prongs being outwardly biased with respect to one another.

13. A securing element as defined in claim 12 wherein the handle joins the two prongs.

14. A method of augmenting a fixation device having a channel extending along an outer surface of the fixation device, the method comprising inserting at least one elongate member of a securing element into the channel such that the elongate member allows the fixation device to resist rotation.

15. A method as defined in aim 14, wherein the securing element is removable.

16. A method as defined in claim 14 or 15, wherein the elongate member comprises a prong adapted to extend along a substantial portion of the channel.

17. A method as defined in any one of claims 14-16, wherein the securing element comprises an elongate member and a handle, and wherein on insertion of the elongate member into the channel, the handle remains outside of the channel.

18. A method as defined in any one of claims 14-17 wherein the securing element comprises two or more prongs extending from a handle, the prongs being biased outwardly with respect to one another.
